(19) United States
(12) Patent Application Publication
(10) Pub. No.: US 2024/0299165 A1
(43) Pub. Date: Sep. 12, 2024

(54) METHODS AND DEVICES FOR FOLDING LEAFLETS OF A HOST VALVE USING COMMISSURES OF A PROSTHETIC VALVE

(71) Applicant: EDWARDS LIFESCIENCES CORPORATION, Irvine, CA (US)

(72) Inventors: Anatoly Dvorsky, Haifa (IL); Ofir Witzman, Harish (IL); David Maimon, Atlit (IL)

(21) Appl. No.: 18/664,220

(22) Filed: May 14, 2024

Related U.S. Application Data
(63) Continuation of application No. PCT/US2022/050042, filed on Nov. 16, 2022.
(60) Provisional application No. 63/281,430, filed on Nov. 19, 2021.

(51) Int. Cl. A61F 2/24

(52) U.S. Cl. CPC A61F 2/2418; A61F 2/2433; A61F 2220/0016; A61F 2220/0075

(57) ABSTRACT

Method and devices for displacing leaflets of an existing valvular structure at an aortic valve position away from coronary ostia of an aorta are disclosed. An example method can include implanting a prosthetic heart valve within a native valve or previously implanted heart valve such that a commissure of the prosthetic heart valve is circumferentially aligned with a mid-portion of a leaflet of the native or previously implanted heart valve. The method further includes, during radially expanding the prosthetic heart valve into the native or previously implanted heart valve, applying pressure to the mid-portion of the leaflet with the commissure such that the leaflet is displaced away from the commissure and toward an inflow end of the prosthetic heart valve.

### CROSS-REFERENCE TO RELATED APPLICATIONS

This application is a continuation of PCT Application No. PCT/US2022/050042, filed Nov. 16, 2022, which claims the benefit of U.S. Provisional Patent Application No. 63/281,430, filed Nov. 19, 2021, each of which is incorporated by reference herein in its entirety.

### FIELD

The present disclosure relates to prosthetic heart valves, and to methods and devices for implanting a prosthetic heart valve within an existing valvular structure such that its commissures displace or fold the leaflets of the existing valvular structure during radial expansion of the prosthetic heart valve.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

As surgical approaches for valve replacement become available for younger patients, patient lifetime may exceed the corresponding lifetime of the implanted prosthetic valve. Valve-in-valve (ViV) procedures have been developed to mount a new prosthetic valve within the previously-implanted prosthetic valve. However, such procedures may pose a risk of coronary artery obstruction. In particular, the leaflets of the previously-implanted prosthetic valve may block the coronary artery ostia or otherwise inhibit blood flow through the frame of the new prosthetic valve to the coronary artery ostia. A similar problem may occur when a prosthetic valve is percutaneously expanded within a native heart valve, for example, when the native leaflets are displaced outward toward the coronary ostia.

### SUMMARY

Described herein are examples of methods and devices for folding and/or displacing leaflets of a heart valve to avoid, or at least reduce the risk of, obstruction of the coronary ostia. In some examples, a part of a leaflet (or parts of leaflets) of an existing valvular structure is translated away from and held distal to the coronary ostia by one or more external features of a prosthetic heart valve during radial expansion of the prosthetic heart valve within the existing valvular structure (e.g., a native heart valve or a previously implanted prosthetic heart valve). In some examples, a part of a leaflet (or parts of leaflets) is folded over itself and/or held distal to the coronary ostia by a commissure or an element attached to a commissure of the prosthetic heart valve which protrudes outward from an outer surface of a frame of the prosthetic heart valve.

In some examples, a method includes implanting a prosthetic heart valve within a native valve or previously implanted prosthetic heart valve such that at least one commissure of the prosthetic heart valve is circumferentially aligned with a mid-portion of a leaflet of the native valve or previously implanted prosthetic heart valve, the mid-portion of the leaflet disposed between two adjacent commissures of the native valve or previously implanted prosthetic heart valve. The method further includes, during radially expanding the prosthetic heart valve into the native valve or previously implanted prosthetic heart valve, applying pressure to the mid-portion of the leaflet with the at least one commissure such that the leaflet of the native valve or previously implanted prosthetic heart valve is displaced away from the at least one commissure and toward an inflow end of the prosthetic heart valve.

In some examples, a prosthetic heart valve includes a radially expandable annular frame comprising an inflow end and an outflow end, wherein a central longitudinal axis of the frame extends between the inflow end and the outflow end; a plurality of leaflets arranged within the frame; and at least one commissure comprising commissure tabs of two adjacent leaflets of the plurality of leaflets connected to each other, the at least one commissure coupled to the frame with at least a portion of the at least one commissure protruding radially outward from an outer surface of the frame, relative to the central longitudinal axis. The prosthetic heart valve further includes a protruding member coupled to the at least one commissure and extending radially outward from the commissure, the protruding member configured to engage a leaflet of a host valve in which the prosthetic heart valve is implanted.

In some examples, a method includes advancing a distal end portion of a delivery apparatus toward a native aortic valve of a heart, wherein a prosthetic heart valve is radially compressed around a valve mounting portion of the delivery apparatus. The method further includes positioning the radially compressed prosthetic heart valve within the native aortic valve or a previously implanted prosthetic heart valve implanted within the native aortic valve such that commissures of the radially compressed prosthetic heart valve are circumferentially aligned with mid-portions of leaflets of the native aortic valve or previously implanted prosthetic heart valve at a location proximal to free edges of the leaflets, wherein the mid-portions of the leaflets are disposed between adjacent commissures of the native aortic valve or previously implanted prosthetic heart valve. The method further includes radially expanding the prosthetic heart valve using the delivery apparatus and, during the radially expanding, engaging and displacing the free edges of the leaflets of the native aortic valve or previously implanted prosthetic heart valve toward an inflow end of the prosthetic heart valve with the commissures of the prosthetic heart valve as it radially expands.

The above methods can be performed on a living animal or on a simulation, such as on a cadaver, cadaver heart, anthropomorphic ghost, simulator (e.g., with body parts, heart, tissue, etc. being simulated), or the like.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a perspective view of a prosthetic heart valve, according to an example.

FIG. 2 is a side view of an example of a delivery apparatus configured to deliver and implant a radially expandable prosthetic heart valve at an implantation site.

FIG. 3 is a perspective view of a prosthetic heart valve mounted within a host surgical heart valve.

FIG. 4 shows a side view of a prosthetic heart valve implanted in the native aortic valve annulus.

FIG. 5 illustrates an exemplary host heart valve that has undergone laceration of its leaflet, longitudinally along the leaflet.

FIG. 6 is a perspective view of a prosthetic heart valve implanted within a host heart valve, where commissures of the prosthetic heart valve displace free edges of the leaflets toward an inflow end of the prosthetic heart valve during radial expansion of the prosthetic heart valve.

FIG. 7 is a schematic showing a side view of a prosthetic heart valve comprising a frame and a plurality of leaflets, where a commissure of the prosthetic heart valve extends radially outward from the frame and an additional protruding member is attached to the commissure, exterior to the frame.

FIG. 8 is a perspective view of a prosthetic heart valve, according to an example.

FIG. 9 is a perspective view of a prosthetic heart valve, according to an example.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

### Overview of the Disclosed Technology

As introduced above, implanted prosthetic valves may need to be replaced in a patient after a period of time. Valve-in-valve (ViV) procedures, which include mounting a new prosthetic valve within the previously-implanted prosthetic valve, may pose a risk of coronary artery obstruction. In particular, the leaflets of the previously-implanted prosthetic valve may block the coronary artery ostia or otherwise inhibit blood flow through the frame of the new prosthetic valve to the coronary artery ostia. A similar problem may occur when a prosthetic valve is percutaneously expanded within a native heart valve, for example, when the native leaflets are displaced outward toward the coronary ostia. Existing methods, which rely on lacerating existing leaflets, require high spatial precision and surgical skill. Moreover, portions of the lacerated leaflet may still act to partially or completely obstruct the coronary ostia.

Described herein are methods and devices for displacing leaflets of a heart valve (e.g., a native heart valve or a previously implanted heart valve). In some examples, the heart valve is at the aortic position and the displacing is effective to avoid, or at least reduce the risk of, obstructing blood flow to the coronary arteries. In some examples, a part of a leaflet (or parts of leaflets) is displaced and held distal to (upstream of) the coronary ostia by one or more external features of a prosthetic heart valve during radial expansion of the prosthetic heart valve within an existing valvular structure (e.g., native heart valve or a previously implanted prosthetic heart valve). In some examples the external feature of the prosthetic heart valve is a portion of a commissure of the prosthetic heart valve that protrudes radially outward from a frame of the prosthetic heart valve. In some examples, the external feature is a radially protruding member that is attached to the commissure of the prosthetic heart valve and is configured to push and hold the leaflets of the heart valve toward an inflow end of the prosthetic heart valve and away from the coronary ostia.

In some examples, methods for implanting the prosthetic heart valve in the native or previously implanted heart valve include implanting the prosthetic heart valve within the native or previously implanted heart valve such that at least one commissure of the prosthetic heart valve is circumferentially aligned with a mid-portion of a leaflet of the native or previously implanted heart valve, the mid-portion of the leaflet disposed between two adjacent commissures of the native or previously implanted heart valve. As such, during radial expansion of the prosthetic heart valve, the commissures or radially protruding members attached to the commissures of the prosthetic heart valve will apply a pressure that displaces the leaflets of the native or previously implanted heart valve away from the commissure and toward an inflow end of the prosthetic heart valve, thereby preventing or at least reducing obstruction of blood flow to the coronary arteries.

In some examples, the heart valve is at a position other than the aortic position, e.g., the pulmonary, tricuspid, or mitral positions.

### Examples of the Disclosed Technology

Prosthetic valves disclosed herein can be radially compressible and expandable between a radially compressed state and a radially expanded state. Thus, the prosthetic valves can be crimped on or retained by an implant delivery apparatus in the radially compressed state during delivery, and then expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

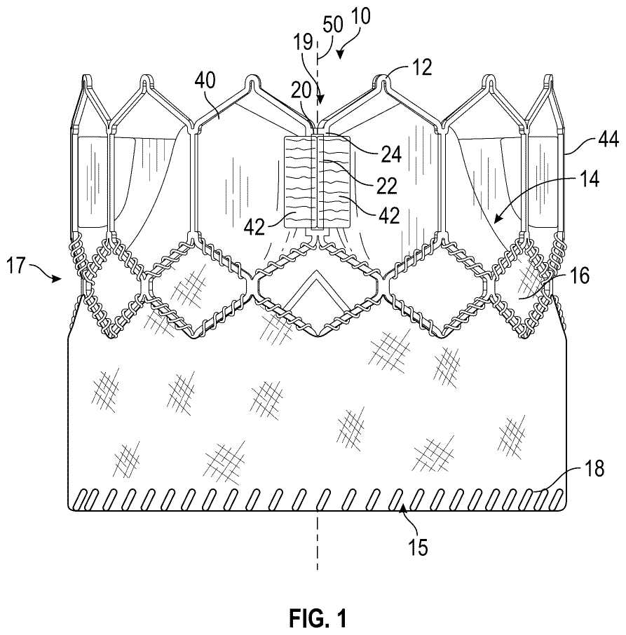

FIG. 1 shows an exemplary prosthetic valve 10, according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In some examples, the disclosed prosthetic valves can be implanted within a docking device implanted within or at the native mitral valve, such as disclosed in PCT Publication No. WO2020/247907, which is incorporated herein by reference. In some examples, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

The prosthetic valve 10 comprises four main components: a stent or frame 12, a valvular structure 14, an inner skirt 16, and a perivalvular outer sealing member or outer skirt 18. The prosthetic valve 10 can have an inflow end portion 15 (also referred to herein as an “inflow end”), an intermediate portion 17, and an outflow end portion 19. The inner skirt 16 can be arranged on and/or coupled to an inner surface of the frame 12, while the outer skirt 18 can be arranged on and/or coupled to an outer surface of the frame 12.

The valvular structure 14 can comprise three leaflets 40, collectively forming a leaflet structure, which can be arranged to collapse in a tricuspid arrangement, although in some instances there can be greater or fewer number of leaflets (e.g., one or more leaflets 40). The leaflets 40 can be secured to one another at their adjacent sides to form commissures 22 of the valvular (e.g., leaflet) structure 14. The lower edge of valvular structure 14 can have an undulating, curved scalloped shape and can be secured to the inner skirt 16 by sutures (not shown). In some examples, the leaflets 40 can be formed of pericardial tissue (e.g., bovine pericardial tissue), biocompatible synthetic materials, or various other suitable natural or synthetic materials as known in the art and described in U.S. Pat. No. 6,730,118, which is incorporated by reference herein.

The frame 12 can be formed with a plurality of circumferentially spaced slots, or commissure windows 20 that are adapted to mount the commissures 22 of the valvular structure 14 to the frame. For example, the commissure windows 20 can be defined by axially extending window strut portions 24 of the frame 12, which can also be referred to herein as “commissure supports”. Each commissure window 20 is adapted to receive a pair of commissure tabs 42 of a pair of adjacent leaflets 40 arranged into a corresponding commissure 22. As shown in FIG. 1 and described further below, the pair of commissure tabs 42 extend from inside the frame 12, through the commissure window 20, and exterior to the frame 12. For example, as shown in FIG. 1, the commissure tabs 42 can extend over and/or protrude radially outward from an outer surface 44 (radially outward facing surface) of the frame 12, and in particular, outer surfaces of the window strut portions 24.

The frame 12 can be made of any of various suitable plastically-expandable materials (e.g., stainless steel, etc.) or self-expanding materials (e.g., nickel titanium alloy (NiTi), such as nitinol), as known in the art. When constructed of a plastically-expandable material, the frame 12 (and thus the prosthetic valve 10) can be crimped to a radially collapsed configuration on a delivery catheter and then expanded inside a patient by an inflatable balloon or equivalent expansion mechanism. When constructed of a self-expandable material, the frame 12 (and thus the prosthetic valve 10) can be crimped to a radially collapsed configuration and restrained in the collapsed configuration by insertion into a sheath or equivalent mechanism of a delivery catheter. Once inside the body, the prosthetic valve can be advanced from the delivery sheath, which allows the prosthetic valve to expand to its functional size.

Suitable plastically-expandable materials that can be used to form the frame 12 include, without limitation, stainless steel, a biocompatible, high-strength alloys (e.g., a cobalt-chromium or a nickel-cobalt-chromium alloys), polymers, or combinations thereof. In particular examples, frame 12 is made of a nickel-cobalt-chromium-molybdenum alloy, such as MP35N® alloy (SPS Technologies, Jenkintown, Pennsylvania), which is equivalent to UNS R30035 alloy (covered by ASTM F562-02). MP35N® alloy/UNS R30035 alloy comprises 35% nickel, 35% cobalt, 20% chromium, and 10% molybdenum, by weight. Additional details regarding the prosthetic valve 10 and its various components are described in WIPO Patent Application Publication No. WO 2018/222799, which is incorporated herein by reference.

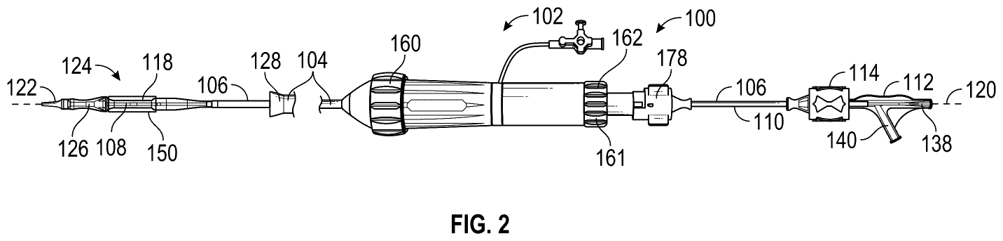

FIG. 2 shows a delivery apparatus 100, according to an example, that can be used to implant an expandable prosthetic heart valve (e.g., the prosthetic heart valve 10 of FIG. 1 and/or any of the prosthetic heart valves described herein). In some examples, the delivery apparatus 100 is specifically adapted for use in introducing a prosthetic valve into a heart.

The delivery apparatus 100 in the illustrated example of FIG. 2 is a balloon catheter comprising a handle 102 and a steerable, outer shaft 104 extending distally from the handle 102. The delivery apparatus 100 can further comprise an intermediate shaft 106 (which also may be referred to as a balloon shaft) that extends proximally from the handle 102 and distally from the handle 102, the portion extending distally from the handle 102 also extending coaxially through the outer shaft 104. Additionally, the delivery apparatus 100 can further comprise an inner shaft 108 extending distally from the handle 102 coaxially through the intermediate shaft 106 and the outer shaft 104 and proximally from the handle 102 coaxially through the intermediate shaft 106.

The outer shaft 104 and the intermediate shaft 106 can be configured to translate (e.g., move) longitudinally, along a central longitudinal axis 120 of the delivery apparatus 100, relative to one another to facilitate delivery and positioning of a prosthetic valve at an implantation site in a patient's body.

The intermediate shaft 106 can include a proximal end portion 110 that extends proximally from a proximal end of the handle 102, to an adaptor 112. A rotatable knob 114 can be mounted on the proximal end portion 110 and can be configured to rotate the intermediate shaft 106 around the central longitudinal axis 120 and relative to the outer shaft 104.

The adaptor 112 can include a first port 138 configured to receive a guidewire therethrough and a second port 140 configured to receive fluid (e.g., inflation fluid) from a fluid source. The second port 140 can be fluidly coupled to an inner lumen of the intermediate shaft 106.

The intermediate shaft 106 can further include a distal end portion that extends distally beyond a distal end of the outer shaft 104 when a distal end of the outer shaft 104 is positioned away from an inflatable balloon 118 of the delivery apparatus 100. A distal end portion of the inner shaft 108 can extend distally beyond the distal end portion of the intermediate shaft 106.

The balloon 118 can be coupled to the distal end portion of the intermediate shaft 106.

In some examples, a distal end of the balloon 118 can be coupled to a distal end of the delivery apparatus 100, such as to a nose cone 122 (as shown in FIG. 2), or to an alternate component at the distal end of the delivery apparatus 100 (e.g., a distal shoulder). An intermediate portion of the balloon 118 can overlay a valve mounting portion 124 of a distal end portion of the delivery apparatus 100 and a distal end portion of the balloon 118 can overly a distal shoulder 126 of the delivery apparatus 100. The valve mounting portion 124 and the intermediate portion of the balloon 118 can be configured to receive a prosthetic heart valve in a radially compressed state. For example, as shown schematically in FIG. 2, a prosthetic heart valve 150 (which can be one of the prosthetic valves described herein) can be mounted around the balloon 118, at the valve mounting portion 124 of the delivery apparatus 100.

The balloon shoulder assembly, including the distal shoulder 126, is configured to maintain the prosthetic heart valve 150 (or other medical device) at a fixed position on the balloon 118 during delivery through the patient's vasculature.

The outer shaft 104 can include a distal tip portion 128 mounted on its distal end. The outer shaft 104 and the intermediate shaft 106 can be translated axially relative to one another to position the distal tip portion 128 adjacent to a proximal end of the valve mounting portion 124, when the prosthetic valve 150 is mounted in the radially compressed state on the valve mounting portion 124 (as shown in FIG. 2) and during delivery of the prosthetic valve to the target implantation site. As such, the distal tip portion 128 can be configured to resist movement of the prosthetic valve 150 relative to the balloon 118 proximally, in the axial direction, relative to the balloon 118, when the distal tip portion 128 is arranged adjacent to a proximal side of the valve mounting portion 124.

An annular space can be defined between an outer surface of the inner shaft 108 and an inner surface of the intermediate shaft 106 and can be configured to receive fluid from a fluid source via the second port 140 of the adaptor 112. The annular space can be fluidly coupled to a fluid passageway formed between the outer surface of the distal end portion of the inner shaft 108 and an inner surface of the balloon 118. As such, fluid from the fluid source can flow to the fluid passageway from the annular space to inflate the balloon 118 and radially expand and deploy the prosthetic valve 150.

An inner lumen of the inner shaft can be configured to receive a guidewire therethrough, for navigating the distal end portion of the delivery apparatus 100 to the target implantation site.

The handle 102 can include a steering mechanism configured to adjust the curvature of the distal end portion of the delivery apparatus 100. In the illustrated example, for example, the handle 102 includes an adjustment member, such as the illustrated rotatable knob 160, which in turn is operatively coupled to the proximal end portion of a pull wire. The pull wire can extend distally from the handle 102 through the outer shaft 104 and has a distal end portion affixed to the outer shaft 104 at or near the distal end of the outer shaft 104. Rotating the knob 160 can increase or decrease the tension in the pull wire, thereby adjusting the curvature of the distal end portion of the delivery apparatus 100. Further details on steering or flex mechanisms for the delivery apparatus can be found in U.S. Pat. No. 9,339,384, which is incorporated by reference herein.

The handle 102 can further include an adjustment mechanism 161 including an adjustment member, such as the illustrated rotatable knob 162, and an associated locking mechanism including another adjustment member, configured as a rotatable knob 178. The adjustment mechanism 161 is configured to adjust the axial position of the intermediate shaft 106 relative to the outer shaft 104 (e.g., for fine positioning at the implantation site). Further details on the delivery apparatus 100 can be found in PCT Application No. PCT/US2021/047056, which is incorporated by reference herein.

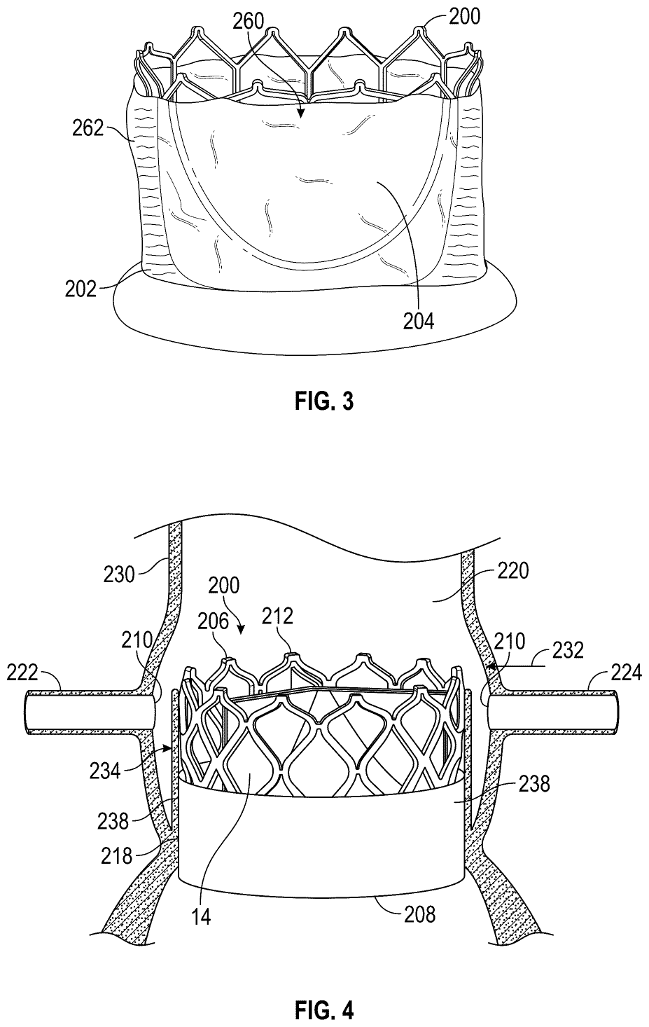

For an existing implanted prosthetic valve, the valvular structure may naturally degrade over time thereby requiring repair or replacement in order to maintain adequate heart functions. In a Valve-in-Valve (ViV) procedure, a new prosthetic heart valve (also referred to herein as a “guest valve”) is mounted within the existing, degrading prosthetic heart valve (also referred to herein as a “host valve”) in order to restore proper function. FIG. 3 shows an example of a prosthetic heart valve 200 (guest valve) mounted within a surgical host heart valve 202. As shown in FIG. 3, the leaflets 204 of the host heart valve 202 can cover and be pressed against a frame of the prosthetic heart valve 200. In some examples, the host heart valve 202 can be another type of valve such as a native aortic valve or another type of prosthetic heart valve. For example, FIG. 4 is a schematic illustrating the prosthetic heart valve 200 implanted within the native aortic annulus 218 (of a native aortic valve).

ViV procedures for implanting a new prosthetic heart valve in the aortic valve position may pose an increased risk of obstruction of the coronary arteries 222, 224 that branch off from the aorta 220 (FIG. 4). In particular, the mounting of the new prosthetic heart valve 200 within the valvular structure of the existing prosthetic heart valve (as shown in FIG. 3) or the native aortic valve (as shown in FIG. 4) can displace the leaflets of the existing heart valve outwards, thereby obstructing the coronary ostia 210 of the coronary arteries 222, 224 (as shown in FIG. 4 for leaflets 238 of the native aortic valve). Moreover, since the leaflets 238 of the existing heart valve are disposed outside the frame of the new prosthetic valve 200, they may cover external surfaces of the frame 212, thereby creating a substantially impermeable tubular structure that occludes openings 234 in frame 212 (FIG. 4). In some patient anatomies (e.g., when the outflow end 206 of the prosthetic heart valve 200 is at the sinotubular (STJ) level 232 and the diameter of the valve 200 is similar to the STJ diameter such that the frame 212 touches or is very close to the aortic wall 230 at the STJ level 232), the leaflets of the existing valve structure may reduce the ability for future access into the coronary arteries 222, 224 or perfusion through the valve frame 212 to the coronary arteries 222, 224 during the diastole phase of the cardiac cycle. Similar problems may occur in some patient anatomies when a prosthetic heart valve 200 is percutaneously expanded within a native valve, displacing the native leaflets 238 outward toward the coronary ostia (as shown in FIG. 4).

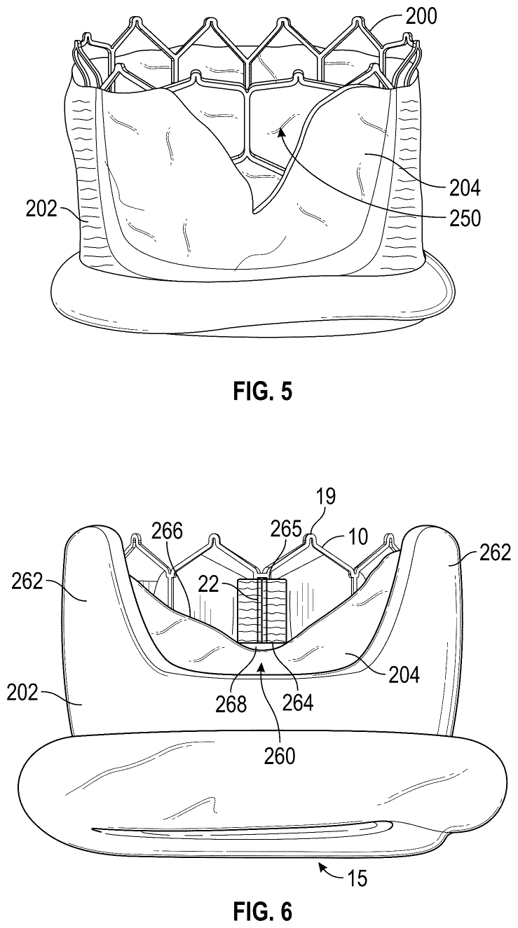

To avoid obstruction of blood flow to the coronary arteries 222, 224, the leaflets of the existing or host heart valve (whether a native aortic valve or a previously implanted prosthetic valve), can be lacerated from based to tip (longitudinally cut) prior to implanting the guest or new prosthetic heart valve. For example, as shown in FIG. 5, the leaflets 204 of the exemplary host heart valve 202 can be split longitudinally, thereby creating a gap 250 between the cut portions of the leaflets 204. Though the guest prosthetic heart valve 200 is shown implanted within the host heart valve 202 in FIG. 5, this laceration procedure is traditionally performed prior to implanted the new or guest prosthetic heart valve 200. As a result, there may be a risk of blood flow leakage through the gaps 250 in the cut portions of the leaflets 204 until the guest prosthetic heart valve 200 can be deployed and implanted within the host heart valve 202. Additionally, as can be appreciated, the laceration procedure increases the overall complexity and cost of a valve replacement procedure.

Thus, instead of lacerating the leaflets of the existing or host heart valve, the new prosthetic heart valve can be configured and implanted such that its commissures (which at least partially protrude radially outward from the frame of the prosthetic heart valve, on an exterior of the frame) exert pressure against the native leaflets of the host heart valve and displace the native leaflets away from the prosthetic heart valve commissures and toward an inflow end of the prosthetic heart valve, as the prosthetic heart valve is radially expanded within the host heart valve. As a result, the native leaflets of the host valve can be translated toward the aortic annulus 218 and away from the coronary ostia 210, thereby reducing or preventing blood flow obstruction to the coronary arteries 222, 224. In some examples, a new prosthetic heart valve can include one or more additional protruding members or elements attached to the prosthetic heart valve commissures on an exterior of the frame of the prosthetic heart valve. Such protruding members can be configured to direct or displace the native (or existing) valve leaflets toward an inflow end of the prosthetic heart valve and/or cut the native (or existing) valve leaflets (similar to as shown in FIG. 5).

As introduced above with reference to FIG. 1, at least a portion of the commissures 22 of the prosthetic heart valve 10 can protrude radially outward from the frame 12, relative to a portion of the frame to which they are connected (e.g., the window strut portions 24). As a result, at least a portion of the commissures 22 is arranged on an outside of the frame 12 and protrudes radially outward from an outer surface of the frame 12, relative to a central longitudinal axis 50 of the frame 12 (FIG. 1).

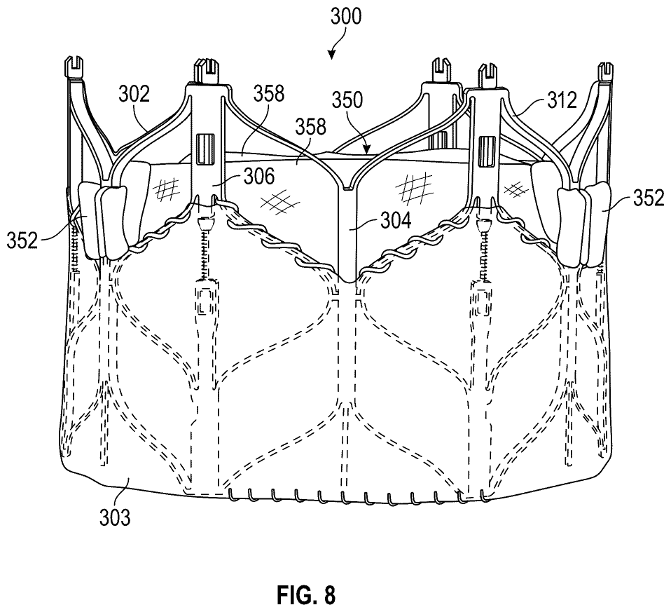

Radially protruding commissures or commissure portions can also be present in other exemplary prosthetic heart valves having a different frame design than frame 12 of prosthetic heart valve 10. For example, FIG. 8 shows a prosthetic heart valve 300 configured as a mechanically expandable prosthetic heart valve. The prosthetic heart valve 300 includes an annular frame 302 and a valvular structure 350 comprising a plurality of leaflets 358 mounted within and to the frame 302. The prosthetic heart valve 300 can also have an optional outer skirt 303 disposed around an outer surface of the frame 302. The frame 302 comprises a plurality of axially extending posts 304 and a plurality of struts 312 connecting the plurality of posts 304. A portion of the posts 304 are actuator mechanisms 306 (which can be referred to expansion and locking mechanisms) which can be used to radially expand and/or radially compress the prosthetic heart valve 300. Commissure tabs of adjacent leaflets 358 can be paired together and extend through commissure windows formed in another portion of the posts 304 (e.g., support posts), thereby forming commissures 352 secured to the frame 302 which protrude radially outward from the frame 302. Additional details on the mechanically expandable prosthetic heart valve 300 can be found in U.S. Provisional Application Nos. 63/224,534 and 63/278,597, both of which are incorporated by reference herein.

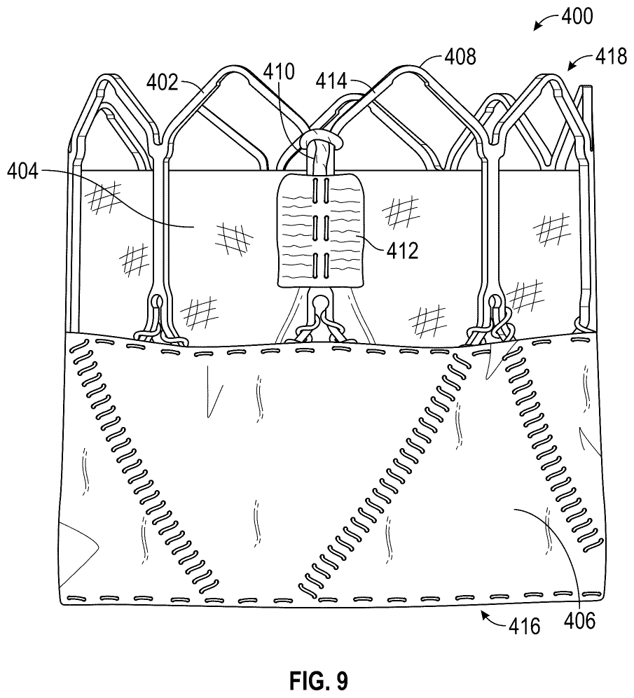

FIG. 9 shows a prosthetic heart valve 400 comprising a radially expandable and/or compressible annular frame 402, a plurality of leaflets 404 mounted within the frame 402, and an outer skirt 406 secured to and around an outer surface of the frame 402. The frame 402 can comprises a plurality of interconnected struts 414 and a plurality of apices 408 that are spaced circumferentially apart around an inflow end portion 416 and an outflow end portion 418 of the frame 402 (only the apices 408 at the outflow end portion 418 are visible in FIG. 9). Each apex 408 is formed at a junction between two angled struts 414 at either the inflow end portion 416 or the outflow end portion 418. The frame 402 comprises a plurality of axially extending posts 410, some of which define commissure windows therein. Commissure tabs of adjacent leaflets 404 can be paired together and extend through the commissure windows, thereby forming commissures 412 secured to the frame 402 which protrude radially outward from the frame 402. In some examples, the prosthetic heart valve 400 can be a balloon expandable valve. Additional details on the prosthetic heart valve 400 can be found in U.S. Provisional Application No. 63/279,096, which is incorporated by reference herein.

During an implantation procedure where the prosthetic heart valve 10 (or another radially expandable prosthetic heart valve, such as any of the prosthetic heart valves described herein) is delivered to and implanted within a host heart valve (either the native valve or a previously implanted prosthetic heart valve), the prosthetic heart valve 10 can be implanted in a specified circumferential position relative to the host heart valve that results in the commissures 22 of the prosthetic heart valve 10 displacing the native leaflets of the host heart valve away from the coronary ostia and toward an inflow end (e.g., inflow end portion 15 shown in FIG. 1) of the prosthetic heart valve 10.

Such an implantation procedure or method is described below with reference to FIG. 2 (showing an exemplary delivery apparatus 100 for a prosthetic heart valve) and FIG. 6 (showing the prosthetic heart valve 10 implanted within an exemplary host heart valve which, in this example, is the surgical host heart valve 202).

The implantation procedure begins by mounting, in a radially compressed configuration or state, the prosthetic heart valve 10 on a valve mounting portion of a distal end portion of the delivery apparatus (e.g., valve mounting portion 124 of delivery apparatus 100 shown in FIG. 2). The distal end portion of the delivery apparatus can then be advanced toward the aortic valve (and the host heart valve 202 implanted therein), or another target heart valve. Upon reaching the implantation site (e.g., the aorta and aortic valve), the radially compressed prosthetic heart valve 10 can be directed into an interior of the host heart valve 202. The delivery apparatus can then be used to manipulate (e.g., rotate) the radially compressed prosthetic heart valve 10 into a target rotational position relative to the host heart valve 202 such that the commissures 22 of the prosthetic heart valve 10 are circumferentially aligned with a middle or mid-portion 260 of the leaflets 204 (shown in FIGS. 3 and 6). The mid-portions 260 of the leaflets 204 are disposed between two adjacent commissures 262 of the host heart valve 202.

Additionally, in some examples, the radially compressed prosthetic heart valve 10 can be axially aligned such that its commissures 22 are positioned slightly proximal to (above in FIG. 6) free edges 266 (between adjacent commissures 262) of the leaflets 204, at or proximate to mid-portions 260 of the leaflets 204.

The prosthetic heart valve 10 can then be radially expanded with the delivery apparatus 100. For example, if the prosthetic heart valve 10 is a balloon expandable valve, the prosthetic heart valve 10 can be radially expanded into a radially expanded configuration or state by inflating a balloon of the delivery apparatus (e.g., inflatable balloon 118 of delivery apparatus 100). In some examples, if the prosthetic heart valve is a self-expanding or mechanically expandable valve, the prosthetic heart valve can be deployed by removing a capsule or sheath covering the radially compressed prosthetic heart valve (in the case of the self-expanding valve) or actuating one or more actuator assemblies configured to mechanically expand the prosthetic heart valve (in the case of the mechanically expandable valve), using the delivery apparatus.

As the prosthetic heart valve 10 is radially expanded within the host heart valve 202, the commissures 22 of the prosthetic heart valve engage the free edges 266 of the leaflets 204 (at or near the mid-portions 260) of the host heart valve 202 and apply a pressure against (e.g., push) the mid-portions 260 of the leaflets 204. This pressure can be a radially outward and/or distally directed pressure that can causes the mid-portions 260 of the leaflets 204 to slide over and off (or at least partially away from) the commissures 22 and be displaced toward the inflow end 15 of the prosthetic heart valve 10.

As shown in FIG. 6, after radial expansion of the prosthetic heart valve 10, one of the leaflets 204 (and in particular the mid-portions 260 of the leaflets 204) of the host heart valve 202 is disposed proximate a first end 264 of the commissure 22 (and away from a second end 265 of the commissure 22 which can be arranged closer to the outflow end 19 of the prosthetic heart valve 10 than the first end 264). The free edge 266 of the leaflet 204 (at or proximate the mid-portion 260 of the leaflet 204) has been displaced vertically away from its commissures 262 and toward the inflow end 15 of the prosthetic heart valve 10. In some examples, this pressure applied by the commissures 22 during radial expansion of the prosthetic heart valve 10 can cause the leaflets 204 of the host heart valve 202 to be folded over themselves (e.g., folded portions 268 of the free edges 266 of the leaflets 204). Further, the radially protruding commissures 22 can hold the leaflets 204 in this folded and displaced configuration, when the prosthetic heart valve 10 is in its radially expanded state.

As a result, when implanted in the aortic valve (e.g., as shown in FIG. 4), the leaflets 204 of the host heart valve 202 can be positioned away from the coronary ostia and toward the inflow end 15 of the prosthetic valve 10 to increase the amount that the outflow cells of the frame 12 are left uncovered by the leaflets 204. As such, blood can more easily flow through the frame 12 of the prosthetic valve 10 to the coronary arteries.

The leaflets 204 of the host heart valve 202 are displaced as described above as a result of the radially protruding commissures 22 of the prosthetic heart valve 10 pushing against the leaflets 204 as the prosthetic heart valve 10 is radially expanded.

In some examples, an additional radially protruding member 270 can be attached to the commissure 22 of the prosthetic heart valve 10 and extend further radially outward from the commissure 22 (and the frame 12).

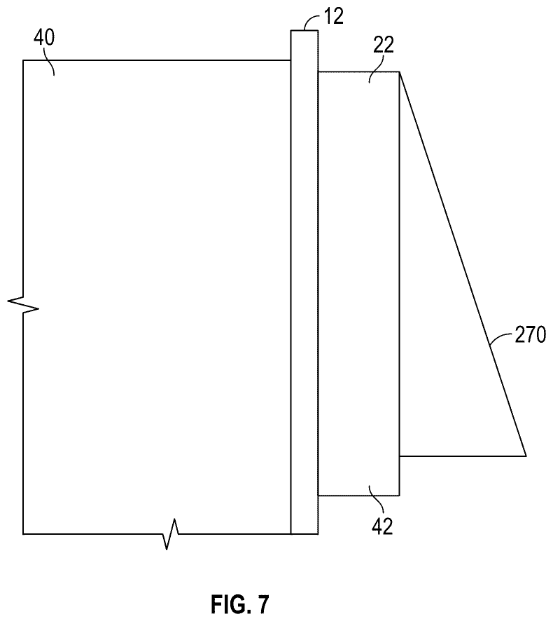

For example, FIG. 7 is a schematic depicting an additional protruding member 270 (which can also be referred to as a “protruding element” or “protruding feature”) attached to the commissure 22 formed from commissure tabs 42 of two adjacent leaflets 40 protruding radially outward through the frame 12 (e.g., through a commissure window 20 as shown in FIG. 1). As shown in FIG. 7, both at least a portion of the commissure 22 and the protruding member 270 are disposed on and extend radially outward beyond an exterior of the frame 12 (e.g., the leaflets 40 are arranged interior to the frame 12 while at least a portion of the commissure 22 and the protruding member 270 are arranged exterior to the frame 12).

In some examples, one or more protruding members 270 can be mounted at other locations on the prosthetic valve 10, such as on the outer surface of the frame 12. For example, protruding members 270 can be mounted to the outer surface of the frame below (upstream of) the commissures 22.

In some examples, the protruding member 270 can be a hook, rod, or other atraumatic member (e.g., rounded, curved, smooth, and/or non-sharp) that is configured to engage the leaflets of the host heart valve and displace them radially outward and distally toward an inflow end 15 of the prosthetic heart valve during radial expansion of the prosthetic heart valve 10. Such a protruding member 270 can be configured to increase engagement with the free edges of the leaflets of the host heart valve, thereby more efficiently displacing them as described above.

In some examples, the protruding member 270 can be blade or other protruding member with a sharp edge that is configured to cut (lacerate) the leaflets of the host heart valve during radial expansion of the prosthetic heart valve (e.g., similar to as shown in FIG. 5). Cutting or lacerating the leaflets of the host heart valve with the protruding member 270 can be advantageous over the traditional laceration procedure described above since the cutting would be performed during radial expansion of the prosthetic heart valve (as compared to prior to guest valve implantation). As a result, the possibility of leakage through the cut leaflets can be reduced.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), or the like.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A method comprising: implanting a prosthetic heart valve within a native valve or previously implanted prosthetic heart valve such that at least one commissure of the prosthetic heart valve is circumferentially aligned with a mid-portion of a leaflet of the native valve or previously implanted prosthetic heart valve, the mid-portion of the leaflet disposed between two adjacent commissures of the native valve or previously implanted prosthetic heart valve; and during radially expanding the prosthetic heart valve into the native valve or previously implanted prosthetic heart valve, applying pressure to the mid-portion of the leaflet with the at least one commissure such that the leaflet of the native valve or previously implanted prosthetic heart valve is displaced away from the at least one commissure and toward an inflow end of the prosthetic heart valve.

Example 2. The method of any example herein, particularly example 1, wherein the at least one commissure comprises commissure tabs of two adjacent leaflets of a plurality of leaflets of the prosthetic heart valve connected to each other, the at least one commissure coupled to a frame of the prosthetic heart valve with at least a portion of the commissure protruding radially outward from an outer surface of the frame, relative to a central longitudinal axis of the frame.

Example 3. The method of any example herein, particularly example 2, wherein the portion of the commissure protruding radially outward from the frame protrudes radially outward from a commissure support of the frame, and wherein the commissure is attached to the commissure support.

Example 4. The method of any example herein, particularly example 3, wherein the commissure support comprises one or more struts defining a commissure window through which the commissure tabs of the commissure extend from an interior to an exterior of the frame.

Example 5. The method of any example herein, particularly any one of examples 1-4, further comprising, prior to radially expanding the prosthetic heart valve, axially positioning the at least one commissure of the prosthetic heart valve, while the prosthetic heart valve is radially compressed onto a distal end portion of a delivery apparatus, proximal to a free edge of the leaflet at the mid-portion of the leaflet.

Example 6. The method of any example herein, particularly any one of examples 1-5, wherein the implanting the prosthetic heart valve within the native valve or previously implanted prosthetic heart valve includes radially expanding the prosthetic heart valve from a radially compressed state to a radially expanded state, within the native valve or previously implanted prosthetic heart valve, by inflating a balloon of a delivery apparatus around which the prosthetic heart valve is mounted in the radially compressed state.

Example 7. The method of any example herein, particularly any one of examples 1-6, wherein the commissure comprises a protruding member attached thereto and wherein the applying pressure to the mid-portion of the leaflet with the at least one commissure includes applying pressure to the leaflet with the protruding member in direct contact with the leaflet.

Example 8. The method of any example herein, particularly example 7, wherein the protruding member is an atraumatic hook or rod.

Example 9. The method of any example herein, particularly example 7, wherein the protruding member is a blade.

Example 10. The method of any example herein, particularly any one of examples 1-9, wherein the applying the pressure to the mid-portion of the leaflet with the at least one commissure includes applying a radially outward and distally directed pressure to the mid-portion of the leaflet.

Example 11. The method of any example herein, particularly any one of examples 1-10, wherein the native valve is an aortic valve, wherein the previously implanted prosthetic heart valve is implanted in the aortic valve, and wherein the inflow end of the prosthetic heart valve, after radial expansion, is disposed away from coronary ostia of an aorta of a heart.

Example 12. The method of any example herein, particularly any one of examples 1-11, wherein the method is performed on a simulation.

Example 13. The method of any example herein, particularly any one of examples 1-11, wherein the method is performed on a human.

Example 14. A prosthetic heart valve comprising: a radially expandable annular frame comprising an inflow end and an outflow end, wherein a central longitudinal axis of the frame extends between the inflow end and the outflow end; a plurality of leaflets arranged within the frame; at least one commissure comprising commissure tabs of two adjacent leaflets of the plurality of leaflets connected to each other, the at least one commissure coupled to the frame with at least a portion of the at least one commissure protruding radially outward from an outer surface of the frame, relative to the central longitudinal axis; and a protruding member coupled to the at least one commissure and extending radially outward from the commissure, the protruding member configured to engage a leaflet of a host valve in which the prosthetic heart valve is implanted.

Example 15. The prosthetic heart valve of any example herein, particularly example 14, wherein the protruding member is a hook or rod configured to push the leaflet of the host valve outward and toward the inflow end of the prosthetic heart valve as the prosthetic heart valve is radially expanded from a radially compressed configuration to a radially expanded configuration.

Example 16. The prosthetic heart valve of any example herein, particularly example 14, wherein the protruding member is a blade configured to cut the leaflet of the host valve as the prosthetic heart valve is radially expanded from a radially compressed configuration to a radially expanded configuration.

Example 17. The prosthetic heart valve of any example herein, particularly any one of examples 14-16, wherein the protruding member protrudes further radially outward than at least the portion of the commissure that protrudes radially outward from the frame.

Example 18. The prosthetic heart valve of any example herein, particularly any one of examples 14-17, wherein the commissure is attached to a commissure support of the frame and wherein the portion of the commissure that protrudes radially outward from the outer surface of the frame protrudes radially outward from an outer surface of the commissure support.

Example 19. The prosthetic heart valve of any example herein, particularly example 18, wherein the commissure support comprises one or more axially extending struts defining a commissure window through which the commissure tabs of the commissure extend from an interior to an exterior of the frame.

Example 20. The prosthetic heart valve of any example herein, particularly any one of examples 14-19, further comprising an outer skirt disposed around an outside of the frame at the inflow end of the frame.

Example 21. A method comprising: advancing a distal end portion of a delivery apparatus toward a native aortic valve of a heart, wherein a prosthetic heart valve is radially compressed around a valve mounting portion of the delivery apparatus; positioning the radially compressed prosthetic heart valve within the native aortic valve or a previously implanted prosthetic heart valve implanted within the native aortic valve such that commissures of the radially compressed prosthetic heart valve are circumferentially aligned with mid-portions of leaflets of the native aortic valve or previously implanted prosthetic heart valve at a location proximal to free edges of the leaflets, wherein the mid-portions of the leaflets are disposed between adjacent commissures of the native aortic valve or previously implanted prosthetic heart valve; and radially expanding the prosthetic heart valve using the delivery apparatus and, during the radially expanding, engaging and displacing the free edges of the leaflets of the native aortic valve or previously implanted prosthetic heart valve toward an inflow end of the prosthetic heart valve with the commissures of the prosthetic heart valve as it radially expands.

Example 22. The method of any example herein, particularly example 21, wherein the displacing the free edges of the leaflets includes folding the free edges of the leaflets of the native aortic valve or previously implanted prosthetic heart valve over themselves and away from the commissures of the prosthetic heart valve.

Example 23. The method of any example herein, particularly either example 21 or example 22, wherein radially expanding the prosthetic heart valve using the delivery apparatus includes inflating a balloon of the delivery apparatus around which the radially compressed prosthetic heart valve is mounted.

Example 24. The method of any example herein, particularly any one of examples 21-23, wherein the displacing the free edges of the leaflets includes displacing and holding the free edges of the leaflets of the native aortic valve or previously implanted prosthetic heart valve away from coronary ostia in an aorta of the heart.

Example 25. The method of any example herein, particularly any one of examples 21-24, wherein the method is performed on a simulation.

Example 26. The method of any example herein, particularly any one of examples 21-24, wherein the method is performed on a human.

Example 27. A method of treating a heart on a simulation, comprising: implanting a prosthetic heart valve within a native valve or previously implanted prosthetic heart valve such that at least one commissure of the prosthetic heart valve is circumferentially aligned with a mid-portion of a leaflet of the native valve or previously implanted prosthetic heart valve, the mid-portion of the leaflet disposed between two adjacent commissures of the native valve or previously implanted prosthetic heart valve; and during radially expanding the prosthetic heart valve into the native valve or previously implanted prosthetic heart valve, applying pressure to the mid-portion of the leaflet with the at least one commissure such that the leaflet of the native valve or previously implanted prosthetic heart valve is displaced away from the at least one commissure and toward an inflow end of the prosthetic heart valve.

Example 28. The method of any example herein, particularly example 27, wherein the at least one commissure comprises commissure tabs of two adjacent leaflets of a plurality of leaflets of the prosthetic heart valve connected to each other, the at least one commissure coupled to a frame of the prosthetic heart valve with at least a portion of the commissure protruding radially outward from an outer surface of the frame, relative to a central longitudinal axis of the frame.

Example 29. The method of any example herein, particularly example 28, wherein the portion of the commissure protruding radially outward from the frame protrudes radially outward from a commissure support of the frame, and wherein the commissure is attached to the commissure support.

Example 30. The method of any example herein, particularly example 29, wherein the commissure support comprises one or more struts defining a commissure window through which the commissure tabs of the commissure extend from an interior to an exterior of the frame.

Example 31. The method of any example herein, particularly any one of examples 27-30, further comprising, prior to radially expanding the prosthetic heart valve, axially positioning the at least one commissure of the prosthetic heart valve, while the prosthetic heart valve is radially compressed onto a distal end portion of a delivery apparatus, proximal to a free edge of the leaflet at the mid-portion of the leaflet.

Example 32. The method of any example herein, particularly any one of examples 27-31, wherein the implanting the prosthetic heart valve within the native valve or previously implanted prosthetic heart valve includes radially expanding the prosthetic heart valve from a radially compressed state to a radially expanded state, within the native valve or previously implanted prosthetic heart valve, by inflating a balloon of a delivery apparatus around which the prosthetic heart valve is mounted in the radially compressed state.

Example 33. The method of any example herein, particularly any one of examples 27-32, wherein the commissure comprises a protruding member attached thereto and wherein the applying pressure to the mid-portion of the leaflet with the at least one commissure includes applying pressure to the leaflet with the protruding member in direct contact with the leaflet.

Example 34. The method of any example herein, particularly example 33, wherein the protruding member is an atraumatic hook or rod.

Example 35. The method of any example herein, particularly example 33, wherein the protruding member is a blade.

Example 36. The method of any example herein, particularly any one of examples 27-35, wherein the applying the pressure to the mid-portion of the leaflet with the at least one commissure includes applying a radially outward and distally directed pressure to the mid-portion of the leaflet.

Example 37. The method of any example herein, particularly any one of examples 27-36, wherein the native valve is an aortic valve, wherein the previously implanted prosthetic heart valve is implanted in the aortic valve, and wherein the inflow end of the prosthetic heart valve, after radial expansion, is disposed away from coronary ostia of an aorta of a heart.

Example 39. A method of treating a heart on a simulation, comprising: advancing a distal end portion of a delivery apparatus toward a native aortic valve of a heart, wherein a prosthetic heart valve is radially compressed around a valve mounting portion of the delivery apparatus; positioning the radially compressed prosthetic heart valve within the native aortic valve or a previously implanted prosthetic heart valve implanted within the native aortic valve such that commissures of the radially compressed prosthetic heart valve are circumferentially aligned with mid-portions of leaflets of the native aortic valve or previously implanted prosthetic heart valve at a location proximal to free edges of the leaflets, wherein the mid-portions of the leaflets are disposed between adjacent commissures of the native aortic valve or previously implanted prosthetic heart valve; and radially expanding the prosthetic heart valve using the delivery apparatus and, during the radially expanding, engaging and displacing the free edges of the leaflets of the native aortic valve or previously implanted prosthetic heart valve toward an inflow end of the prosthetic heart valve with the commissures of the prosthetic heart valve as it radially expands.

Example 40. The method of any example herein, particularly example 39, wherein the displacing the free edges of the leaflets includes folding the free edges of the leaflets of the native aortic valve or previously implanted prosthetic heart valve over themselves and away from the commissures of the prosthetic heart valve.

Example 41. The method of any example herein, particularly either example 39 or example 40, wherein radially expanding the prosthetic heart valve using the delivery apparatus includes inflating a balloon of the delivery apparatus around which the radially compressed prosthetic heart valve is mounted.

Example 42. The method of any example herein, particularly any one of examples 39-41, wherein the displacing the free edges of the leaflets includes displacing and holding the free edges of the leaflets of the native aortic valve or previously implanted prosthetic heart valve away from coronary ostia in an aorta of the heart.

Example 43. A method comprising sterilizing the prosthetic heart valve, apparatus, and/or assembly of any example.

Example 44. A prosthetic heart valve of any one of examples 1-42, wherein the prosthetic heart valve is sterilized.

Example 45. The method of any example herein, further comprising sterilizing the prosthetic heart valve prior to an implantation procedure.

The features described herein with regard to any example can be combined with other features described in any one or more of the other examples, unless otherwise stated. For example, any one or more of the features of prosthetic heart valve can be combined with any one or more features of another prosthetic heart valve.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A method comprising:
implanting a prosthetic heart valve within a native valve or previously implanted prosthetic heart valve such that at least one commissure of the prosthetic heart valve is circumferentially aligned with a mid-portion of a leaflet of the native valve or previously implanted prosthetic heart valve, the mid-portion of the leaflet disposed between two adjacent commissures of the native valve or previously implanted prosthetic heart valve; and
during radially expanding the prosthetic heart valve into the native valve or previously implanted prosthetic heart valve, applying pressure to the mid-portion of the leaflet with the at least one commissure such that the leaflet of the native valve or previously implanted prosthetic heart valve is displaced away from the at least one commissure and toward an inflow end of the prosthetic heart valve.

2. The method of claim 1, wherein the at least one commissure comprises commissure tabs of two adjacent leaflets of a plurality of leaflets of the prosthetic heart valve connected to each other, the at least one commissure coupled to a frame of the prosthetic heart valve with at least a portion of the commissure protruding radially outward from an outer surface of the frame, relative to a central longitudinal axis of the frame.

3. The method of claim 2, wherein the portion of the commissure protruding radially outward from the frame protrudes radially outward from a commissure support of the frame, and wherein the commissure is attached to the commissure support.

4. The method of claim 3, wherein the commissure support comprises one or more struts defining a commissure window through which the commissure tabs of the commissure extend from an interior to an exterior of the frame.

5. The method of claim 1, further comprising, prior to radially expanding the prosthetic heart valve, axially positioning the at least one commissure of the prosthetic heart valve, while the prosthetic heart valve is radially compressed onto a distal end portion of a delivery apparatus, proximal to a free edge of the leaflet at the mid-portion of the leaflet.

6. The method of claim 1, wherein the implanting the prosthetic heart valve within the native valve or previously implanted prosthetic heart valve includes radially expanding the prosthetic heart valve from a radially compressed state to a radially expanded state, within the native valve or previously implanted prosthetic heart valve, by inflating a balloon of a delivery apparatus around which the prosthetic heart valve is mounted in the radially compressed state.

7. The method of claim 1, wherein the commissure comprises a protruding member attached thereto, and wherein the applying pressure to the mid-portion of the leaflet with the at least one commissure includes applying pressure to the leaflet with the protruding member in direct contact with the leaflet.

8. The method of claim 7, wherein the protruding member is an atraumatic hook or rod.

9. The method of claim 7, wherein the protruding member is a blade.

10. The method of claim 1, wherein the applying the pressure to the mid-portion of the leaflet with the at least one commissure includes applying a radially outward and distally directed pressure to the mid-portion of the leaflet.

11. The method of claim 1, wherein the native valve is an aortic valve, wherein the previously implanted prosthetic heart valve is implanted in the aortic valve, and wherein the inflow end of the prosthetic heart valve, after radial expansion, is disposed away from coronary ostia of an aorta of a heart.

12. The method of claim 1, wherein the method is performed on a simulation.

13. A prosthetic heart valve comprising:
a radially expandable annular frame comprising an inflow end and an outflow end, wherein a central longitudinal axis of the frame extends between the inflow end and the outflow end;
a plurality of leaflets arranged within the frame;
at least one commissure comprising commissure tabs of two adjacent leaflets of the plurality of leaflets connected to each other, the at least one commissure coupled to the frame with at least a portion of the at least one commissure protruding radially outward from an outer surface of the frame, relative to the central longitudinal axis; and
a protruding member coupled to the at least one commissure and extending radially outward from the commissure, the protruding member configured to engage a leaflet of a host valve in which the prosthetic heart valve is implanted.

14. The prosthetic heart valve of claim 13, wherein the protruding member is a hook or rod configured to push the leaflet of the host valve outward and toward the inflow end of the prosthetic heart valve as the prosthetic heart valve is radially expanded from a radially compressed configuration to a radially expanded configuration.

15. The prosthetic heart valve of claim 13, wherein the protruding member is a blade configured to cut the leaflet of the host valve as the prosthetic heart valve is radially expanded from a radially compressed configuration to a radially expanded configuration.

16. The prosthetic heart valve of claim 13, wherein the protruding member protrudes further radially outward than at least the portion of the commissure that protrudes radially outward from the frame.

17. A method comprising:
advancing a distal end portion of a delivery apparatus toward a native aortic valve of a heart, wherein a prosthetic heart valve is radially compressed around a valve mounting portion of the delivery apparatus;
positioning the radially compressed prosthetic heart valve within the native aortic valve or a previously implanted prosthetic heart valve implanted within the native aortic valve such that commissures of the radially compressed prosthetic heart valve are circumferentially aligned with mid-portions of leaflets of the native aortic valve or previously implanted prosthetic heart valve at a location proximal to free edges of the leaflets, wherein the mid-portions of the leaflets are disposed between adjacent commissures of the native aortic valve or previously implanted prosthetic heart valve; and
radially expanding the prosthetic heart valve using the delivery apparatus and, during the radially expanding, engaging and displacing the free edges of the leaflets of the native aortic valve or previously implanted prosthetic heart valve toward an inflow end of the prosthetic heart valve with the commissures of the prosthetic heart valve as it radially expands.

18. The method of claim 17, wherein the displacing the free edges of the leaflets includes folding the free edges of the leaflets of the native aortic valve or previously implanted prosthetic heart valve over themselves and away from the commissures of the prosthetic heart valve.

19. The method of claim 17, wherein radially expanding the prosthetic heart valve using the delivery apparatus includes inflating a balloon of the delivery apparatus around which the radially compressed prosthetic heart valve is mounted.

20. The method of claim 17, wherein the displacing the free edges of the leaflets includes displacing and holding the free edges of the leaflets of the native aortic valve or previously implanted prosthetic heart valve away from coronary ostia in an aorta of the heart.
